(19) United States
(12) Patent Application Publication
(10) Pub. No.: US 2021/0405035 A1
(43) Pub. Date: Dec. 30, 2021

(54) MULTIFUNCTIONAL POLYMERIC MICROSPHERE/MICROPARTICLE CELL BIOREACTOR SYSTEM AND SORTING PROCESS

(71) Applicant: THE SECANT GROUP, LLC, Telford, PA (US)

(72) Inventors: Jeremy J. HARRIS, Doylestown, PA (US); Peter D. GABRIELE, Frisco, TX (US); Brian GINN, Harleysville, PA (US)

(21) Appl. No.: 17/348,075

(22) Filed: Jun. 15, 2021

Related U.S. Application Data
(60) Provisional application No. 63/046,070, filed on Jun. 30, 2020.

(51) Int. Cl. G01N 33/545; C12N 5/0783

(52) U.S. Cl. CPC G01N 33/545; C12N 5/0636

(57) ABSTRACT

A cell selection and sorting process includes attaching cells of a target cell type to a first set of polymeric beads, washing the chamber through a first filter having a first pore size less than the first bead diameter to retain the first set of polymeric beads and greater than a cell diameter to remove unattached cells, releasing the cells of the target cell type from the first set of polymeric beads, and collecting the cells of the target cell type. A cell modification process includes modifying cells of the target cell type in the chamber. A cell modification system includes a cell modification chamber with entry ports and outlet ports, filters with predetermined pore sized selectably located on the outlet ports, and sets of polymeric beads with predetermined diameters being selected such that the sets of polymeric beads are separable by the filters.

### CROSS-REFERENCE TO RELATED APPLICATIONS

This application claims priority to and the benefit of U.S. Provisional Application No. 63/046,070 filed Jun. 30, 2020, which is hereby incorporated by reference in its entirety.

### FIELD OF THE INVENTION

The present disclosure is generally directed to systems and processes for selecting or sorting cells. More specifically, the present disclosure is directed to cell modification systems and processes including polymeric beads of a co-polymer of glycerol and sebacic acid (PGS) for controlling separation steps.

### BACKGROUND OF THE INVENTION

The ability to select a specific type of cell and sort that specific type of cell from other cells and other biological material is important for many different cell manipulation processes. For example, cell modification processes include cell selection and cell sorting.

Cell modification processes collect and modify cells for a specific purpose. Conventional cell modification processes include multiple steps. Conventional cell modification processes rely on magnetic beads for cell selection, which significantly limits production scale. Additionally, conventional cell modification processes require multiple transfers into different containers of the cells being modified. This takes additional time and requires additional manipulation of the cells in situations where cell health is maximized when cells are minimally manipulated.

Chimeric antigen receptor (CAR) T cells are genetically engineered T cells for immunotherapy that express an artificial receptor, giving the T cells a specific targeting ability. A conventional multi-step cell modification process to produce CAR T cells includes collecting the T cells from a blood sample, activating the T cells, transducing the T cells, expanding the T cells, and collecting the resulting CAR T cells.

Conventional magnetic beads for CAR T cell production can generally only separate out one cell subpopulation at a time, which is a slow process. For example, in a system containing T helper cells, cytotoxic T cells, and natural killer (NK) cells, CAR T cell production processes using conventional magnetic beads would require separation of these three subpopulations of cells one at a time by first magnetically separating out CD4+ cells and then running the remaining mixed cell population through magnetic separation two more times in sequence to separate CD8+ and then CD56+ cells. This takes additional time and requires additional manipulation of the cells in a situation where cell health is maximized when cells are minimally manipulated.

### BRIEF DESCRIPTION OF THE INVENTION

There is a need for a cell modification process and a cell modification system that is more efficient, is scalable, and reduces handling of the cells.

In exemplary embodiments, a cell selection and sorting process includes attaching cells of a target cell type to a first set of polymeric beads having a first bead diameter in a chamber. The process also includes washing the chamber through a first filter having a first pore size less than the first bead diameter to retain the first set of polymeric beads and greater than a cell diameter to remove unattached cells. The process further includes releasing the cells of the target cell type from the first set of polymeric beads. The process finally includes releasing the cells of the target cell type from the second set of polymeric beads and collecting the modified cells of the target cell type.

In exemplary embodiments, a cell modification process includes attaching cells of a target cell type to a first set of polymeric beads having a first bead diameter in a chamber. The process also includes washing the chamber through a first filter having a first pore size less than the first bead diameter to retain the first set of polymeric beads and greater than a cell diameter to remove unattached cells. The process further includes releasing the cells of the target cell type from the first set of polymeric beads. The process yet further includes modifying the cells of the target cell type in the chamber. The process also includes binding the cells of the target cell type to a second set of polymeric beads having a second bead diameter greater than the first bead diameter. The process additionally includes washing the chamber through a second filter having a second pore size less than the second bead diameter to retain the second set of polymeric beads but greater than the first bead diameter to remove the first set of polymeric beads. The process finally includes releasing the modified cells of the target cell type from the second set of polymeric beads and collecting the modified cells of the target cell type.

In exemplary embodiments, a cell modification system includes a cell modification chamber including at least one entry port and at least one outlet port. The cell modification system also includes a plurality of filters selectably located on the at least one outlet port, each of the plurality of filters having a predetermined pore size. The cell modification system further includes a plurality of sets of polymeric beads, each set of polymeric beads having a predetermined diameter, the predetermined diameters being selected such that the plurality of sets of polymeric beads are separable by the plurality of filters.

In exemplary embodiments, a flow-through cell modification system includes a plurality of catch chambers, each catch chamber having a set of catch polymeric beads of a predetermined diameter to catch a plurality of cells of a predetermined cell type. The flow-through cell modification system also includes a plurality of release chambers, the plurality of catch chambers and the plurality of release chambers being alternatingly sequentially arranged. The flow-through cell modification system further includes a plurality of filters, each filter having a predetermined pore size, each filter separating one of the plurality of catch chambers and one of the plurality of release chambers to separate sets of catch polymeric beads.

Various features and advantages of the present invention will be apparent from the following more detailed description, taken in conjunction with the accompanying drawings which illustrate, by way of example, the principles of the invention.

### BRIEF DESCRIPTION OF THE DRAWINGS

FIG. 1 schematically shows a cell modification system for production of CAR T cells in a single containment vessel in an embodiment of the present disclosure.

FIG. 2 schematically shows an activation step for the cell modification system of FIG. 1.

FIG. 3 schematically shows a gene modification step for the cell modification system of FIG. 1.

FIG. 4 schematically shows an expansion step for the cell modification system of FIG. 1.

FIG. 5 schematically shows a collection step for the cell modification system of FIG. 1.

FIG. 6 schematically shows a vertical stack of filters for a cell modification system for production of CAR T cells in a single containment vessel in an embodiment of the present disclosure.

FIG. 7 schematically shows a cell modification system with four quadrants for production of CART cells in an embodiment of the present disclosure.

FIG. 8 schematically shows a cell modification system including a secondary antechamber in an embodiment of the present disclosure.

FIG. 9 schematically shows a cell modification system including multiple ancillary chambers in an embodiment of the present disclosure.

FIG. 10 schematically shows a flow-through cell modification system for production of CAR T cells in an embodiment of the present disclosure.

FIG. 11 shows images of Jurkat cells attached to and released from polymeric beads.

Wherever possible, the same reference numbers will be used throughout the drawings to represent the same parts.

### DETAILED DESCRIPTION OF THE INVENTION

Provided are cell selection and sorting systems and processes using polymeric beads of a co-polymer of glycerol and sebacic acid (PGS) in controlling separation steps. In exemplary embodiments, the cell selection and cell sorting are part of a cell modification system or process. The cells for selection and/or sorting may be cells in suspension or adherent cells.

Embodiments of the present disclosure, for example, in comparison to concepts failing to include one or more of the features disclosed herein, provide a single-use, self-contained, high through-put manufacturing system for cell-based therapies; contain cells within one containment structure for an entire multi-step cell modification manufacturing process; reduce processing stress on cells; produce higher manufactured cell yields; reduce consumables, such as culture bags, tubing, and tubing sets; reduce set-up time; reduce the risk of contamination; rely on the physical dimensions of the microspheres or cells for selection, sorting, separation, and/or collection; or combinations thereof.

In exemplary embodiments, mixed cell populations are separated in a one-step, one-vessel process where the various cell populations are tethered to various sized polymeric beads. For example, large-sized anti-CD4, medium-sized anti-CD8, small-to-medium-sized anti-CD19 beads, and small-sized anti-CD56 beads are used separate out T helper cells, cytotoxic T cells, B cells, and NK cells, respectively. The bead-containing solution can then be passed through a series of filters to selectively separate the cell populations based on bead size.

In exemplary embodiments, a cell selection and sorting system incorporates polymeric microsphere technology to create specifically engineered microspheres.

In some embodiments, the cell selection and sorting system includes a single insertable cartridge, rather than the multiple bags and tubing used in conventional cell modification systems.

In some embodiments, the cell selection and sorting incorporates a flow-through process.

The terms “microsphere” and “bead” are used interchangeably herein.

In some embodiments, the polymeric beads are commercial polymeric beads. Appropriate commercial polymeric beads may include, but are not limited to, magnetic polymeric beads, fluorescent polymeric microbeads, antibody-labeled polymeric microbeads, polystyrene beads, and/or polysaccharide beads.

The polymeric beads may be porous or non-porous.

In exemplary embodiments, the polymeric beads are PGS microspheres formed of a polyester co-polymer of a polyol and an acid. In exemplary embodiments, the polymeric beads are PGS microspheres formed of a co-polymer of glycerol and sebacic acid (PGS). The PGS may be poly(glycerol sebacate), a PGS-urethane (PGSU), a PGS-acrylate (PGSA), or other glycerol ester derivatives.

In addition to cell tethering, the polymeric beads may be used to supplement the cell chamber by being formulated with various agents required during specific steps of the process. For example, the polymeric beads may also be loaded with cell nutrients, cell-specific supplements, buffering agents, antimicrobial agents, oxygen-producing agents, and/or cellular waste chelating/absorption agents.

One utility of PGS is its ability to be customized. For instance, in cases where biodegradability is not a preferred feature of the bead, the PGS polymer may be polymerized to a degree where degradation is stalled to accommodate the temporal process requirements. Furthermore, the active functionality, such as hydroxy groups and carboxyl groups, allows for surface modification that may include anchored or bound docking chemistry to be used for ferrying cells.

These anchor points may be customized to address each specific step of a cell modification process. For example, with a PGS microsphere having available hydroxy and carboxyl groups, the microsphere may be modified with a photolink and antibody to catch (with antibody specificity) and release on command (photolink) via a specific frequency of irradiation.

The catch and release may be based on either a complexation involving coordination-complexing (ion-ion charge-based attraction) or conjugation (a covalent bridging linkage) between at least two molecules, in this case between a biologic and target-docking molecule on a cell or carrier.

The catch and release biologic complexation/conjugation may be facilitated by either exogenous or endogenous chemical energy variables within the proximity of the molecular target docking or bonding location, leading to a connection change or linkage between molecules, either ionic coordination or covalent bonding. Additionally, the catch and release may be based on the complexation or conjugation site being reversible either by chemical or physical energy. In the case of physical, the delinking may be based on introduction of a frequency-specific photo-emission into the absorbing bonding complex.

For instance, the customized PGS microsphere provides the option of integrating a photolinker and an antibody-specific “tether” for “catch and release” via light-initiated release of specific cells following treatments. The photocleavable link, or photolink, may be covalently coupled to a carboxy group of the microsphere at one point and to the antibody at another point. Photoactivation to release at the photolink separates the antibody-bound cell from the microsphere. Pahattuge et al. [“Visible photorelease of liquid biopsy markers following microfluidic affinity-enrichment”, Chem. Commun., Vol. 56, pp. 4098-4101 (2020), incorporated by reference in its entirety herein] describe an appropriate photolink capable of covalently attaching antibodies and subsequently release them with visible light at greater than 90% release in two minutes.

PGS is also inherently antimicrobial and PGS microspheres may provide metabolic enhancement to cells. This is an added advantage for PGS over other polymeric microspheres, such as, for example, polystyrene or polysaccharide beads.

The cell modification process may be any process including multiple steps of attaching cells, removing unattached components between steps, and detaching cells.

In exemplary embodiments, a cell selection and sorting process includes attaching cells of a target cell type to a first set of polymeric beads having a first bead diameter in a chamber, washing the chamber contents through a first filter having a first pore size less than the first bead diameter to retain the first set of polymeric beads, and releasing the cells of the target cell type from the first set of polymeric beads. When the process is a cell modification process, the process also includes modifying the cells of the target cell type in the chamber, binding the cells of the target cell type to a second set of polymeric beads having a second bead diameter greater than the first bead diameter, and washing the chamber through a second filter having a second pore size less than the second bead diameter to retain the second set of polymeric beads but greater than the first bead diameter to remove the first set of polymeric beads. The process further includes releasing the modified cells of the target cell type from the second set of polymeric beads and collecting the modified cells of the target cell type.

In some embodiments, the target cell type is a subpopulation of T cells.

In exemplary embodiments, subpopulations of T cells can be separated from a mixed cell population by exposure to one or more antigens associated with a bead population, which may include, but are not limited to, anti-CD3+anti-CD4 for CD4⁺ T helper cells, anti-CD3+anti-CD8 for cytotoxic T cells, anti-CD3+anti-CTLA-4 for CD4⁺ T regulatory cells, anti-CD3+anti-CD95 for CD8⁺ T memory cells, anti-Vδ1+anti-Vγ9Vδ2 for γδ T cells, or anti-CD3+anti-CD57 for natural killer T cells.

The exposure to combinations of antigens may be sequential or simultaneous. In some embodiments, the exposure includes sequentially exposing cell mixture to a broader sorter antibody bead, for example, CD3 for T cells, and then removal of cells followed by exposure to a second, differing, subpopulation bead, for example, taking the CD3 positive cells and applying them to anti-CD4 beads to pull out T helper cells. In other embodiments, the exposure includes directly pulling out a subpopulation of cells by exposing to beads simultaneously containing multiple antigens, for example, pulling out T helper cells directly from the initial mixed population by using beads with both anti-CD3 and anti-CD4 on the same bead.

In some embodiments, one or more of the following are used with beads to select follicular helper T cells: anti-BTLA, anti-CD3, anti-CD4, anti-CD10, anti-CD40L, anti-CD57, anti-CD84, anti-CD150, anti-CXCR4, anti-CXCR5, anti-ICOS, anti-IL-6Rα, anti-IL-21R, anti-OX40, anti-PD-1. In some embodiments, one or more of the following are used with beads to select Th1 helper T cells: anti-CCR1, anti-CCR5, anti-CD3, anti-CD4, anti-CXCR3, anti-IFNγR1, anti-IFNγR2, anti-IL-12Rβ2, anti-IL-18Rα, anti-IL-27Rα. In some embodiments, one or more of the following are used with beads to select Th2 helper T cells: anti-CCR3, anti-CCR4, anti-CCR8, anti-CD3, anti-CD4, anti-CXCR4, anti-IL4Rα, anti-IL-17Rb, anti-IL-33R, anti-TSLP R. In some embodiments, one or more of the following are used with beads to select Th9 helper T cells: anti-CD3, anti-CD4, anti-IL-4Rα, anti-IL-17Rb, anti-TGF-βRII. In some embodiments, one or more of the following are used with beads to select Th17 helper T cells: anti-CCR4, anti-CCR6, anti-CD3, anti-CD4, anti-IL-1RI, anti-IL-6Rα, anti-IL21R, anti-IL23R, anti-TGF-β RII. In some embodiments, one or more of the following are used with beads to select Th22 helper T cells: anti-CCR4, anti-CCR6, anti-CCR10, anti-CD3, anti-CD4, anti-IL-6Rα, anti-TNF RI. In some embodiments, one or more of the following are used with beads to select regulatory T cells: anti-CD3, anti-CD4, anti-CD5, anti-CD25, anti-CD39, anti-CD62L, anti-CD73, anti-CD103, anti-CD127, anti-CD134, anti-CD223, anti-CTLA-4, anti-follate receptor 4, anti-GITR, anti-LAP, anti-LRRC32, anti-BDCA-4.

In exemplary embodiments, T cells may become activated upon exposure to beads with anti-CD3 and anti-CD28. In some embodiments, T cells may become activated upon exposure to beads with phytohaemagglutinin conjugated or adsorbed to the bead surface.

In exemplary embodiments, subpopulations of B cells can be separated from a mixed cell population by exposure to sequential or simultaneous combinations of one or more antigens associated with a bead population, which may include, but is not limited to, anti-CD19+anti-CD10 for immature B cells, anti-CD19+anti-CD10+anti-CD27+anti-CD24+anti-CD38 for transitional B cells, anti-CD19+anti-CD22 for follicular B cells, anti-CD19+anti-CD69+anti-CD86 for activated B cells, anti-CD19+anti-CD21+anti-CD27 for memory B cells, anti-CD19+anti-CD43 for regulatory B cells, and anti-CD19+anti-CD27+anti-CD38 for plasma cells. In some embodiments, one or more of the following are used with beads to select follicular B cells: anti-CD19, anti-CD20, anti-CD21, anti-CD22, anti-CD23, anti-CD24, anti-CD38, anti-CXCR5, anti-HLA-DR, anti-IgD, anti-IgM, anti-TACI. In some embodiments, one or more of the following are used with beads to select memory B cells: anti-CD19, anti-CD20, anti-CD21, anti-CD23, anti-CD27, anti-CD40, anti-CD80, anti-CD86, anti-CD93, anti-CD95, anti-CD148, anti-HLA-DR, anti-TACI. In some embodiments, one or more of the following are used with beads to select regulatory B cells: anti-CD1d, anti-CD5, anti-CD19, anti-CD20, anti-CD21, anti-CD24, anti-CD27, anti-CD38, anti-CD40, anti-IgD, anti-IgM. In some embodiments, one or more of the following are used with beads to select plasma B cells: anti-BCMA, anti-CD19, anti-CD27, anti-CD38, anti-CD138, anti-CXCR4.

In exemplary embodiments, B cells may become activated upon exposure to beads with anti-CD40. In some embodiments, lipopolysaccharides are conjugated to a bead surface to activate B cells. In other embodiments, bacterial capsular polysaccharides are conjugated to a bead surface.

In exemplary embodiments, other cell types may be used with the bioreactor system containing beads including: dendritic cells, granulocytes, macrophages, monocytes, innate lymphoid cells, myeloid-derived suppressor cells, induced pluripotent stem cells, embryonic stem cells, hematopoietic stem cells, mesenchymal stem cells, neural stem cells.

In some embodiments, one or more of the following are used with beads to select dendritic cells: anti-CD1a, anti-CD1c, anti-CD11b, anti-CD11c, anti-CD14, anti-CD16, anti-CD40, anti-CD80, anti-CD83, anti-CD86, anti-CD123, anti-CD141, anti-CD172a, anti-CD207, anti-CD209, anti-CXCR1, anti-CLEC9a, anti-EpCAM, anti-HLA-DR, anti-IGSF4A, anti-XCR1. In some embodiments, one or more of the following are used with beads to select inflammatory dendritic cells: anti-CD1a, anti-CD1c, anti-CD11b, anti-CD11c, anti-CD14, anti-CD64, anti-CD172a, anti-CD206, anti-HLA-DR. In some embodiments, one or more of the following are used with beads to select epidermal Langerhans dendritic cells: anti-CD1a, anti-CD1c, anti-CD11b, anti-CD11c, anti-CD172a, anti-CD207, anti-E-Cadherin, anti-EpCAM, anti-HLA-DR. In some embodiments, one or more of the following are used with beads to select CD14⁺ dermal dendritic cells: anti-CD1c, anti-CD11c, anti-CD14, anti-CD163, anti-CD209, anti-HLA-DR. In some embodiments, one or more of the following are used with beads to select CD11a⁺ CD1c⁺ dermal dendritic cells: anti-CD1a, anti-CD1c, anti-CD11b, anti-CD11c, anti-CD141, anti-CD172a, anti-HLA-DR. In some embodiments, one or more of the following are used with beads to select CD1a⁺ CD1c⁺ dermal dendritic cells: anti-CD1a, anti-CD1c, anti-CD11b, anti-CD11c, anti-CD141, anti-CD172a, anti-HLA-DR. In some embodiments, one or more of the following are used with beads to select CD1a⁺ CD141⁺ dermal dendritic cells: anti-CD1a, anti-CD1c, anti-CD11b, anti-CD11c, anti-CD141, anti-CD172a, anti-CLEC9a, anti-HLA-DR, anti-IGSF4A, anti-XCR1.

In some embodiments, one or more of the following are used with beads to select basophil cells: anti-CCR3, anti-CD11c, anti-CD22, anti-CD45, anti-CD49b, anti-CD69, anti-CD123, anti-CD203, anti-anti-CRTH-2, HLA-DR. In some embodiments, one or more of the following are used with beads to select eosinophil cells: anti-CCR3, anti-CD11b, anti-CD14, anti-CD15, anti-CD45, anti-CD49d, anti-CD66b, anti-CD125, anti-EMR1, anti-HLA-DR, anti-Siglec-8. In some embodiments, one or more of the following are used with beads to select mast cells: anti-CD11c, anti-CD32, anti-CD33, anti-CD45, anti-CD117, anti-CD123, anti-CD203c, anti-HLA-DR. In some embodiments, one or more of the following are used with beads to select neutrophil cells: anti-CD11b, anti-CD14, anti-CD15, anti-CD16, anti-CD18, anti-CD32, anti-CD33, anti-CD44, anti-CD45, anti-CD62L, anti-CD66b, anti-HLA-DR.

In some embodiments, one or more of the following are used with beads to select group 1 innate lymphoid cells: anti-CD25, anti-CD45, anti-CD49a, anti-CD69, anti-CD117, anti-CD122, anti-CD127, anti-CD161, anti-CXCR3, anti-ICOS, anti-IL-1RI, anti-IL12Rβ2, anti-NKp46. In some embodiments, one or more of the following are used with beads to select group 2 innate lymphoid cells: anti-CD25, anti-CD45, anti-CD90, anti-CD117, anti-CD127, anti-CD161, anti-CRTH-2, anti-ICOS, anti-IL-1RI, anti-IL-12102, anti-IL-17RB, anti-KLG1, anti-Sca-1, anti-ST2, anti-TSLP R. In some embodiments, one or more of the following are used with beads to select group 3 innate lymphoid cells: anti-CCR6, anti-CD25, anti-CD45, anti-CD90, anti-CD117, anti-CD127, anti-IL-1RI, anti-IL12102, anti-IL-23R, anti-CD56, anti-NKp44, anti-NKp46, anti-Sca-1. In some embodiments, one or more of the following are used with beads to select natural killer cells: anti-CD56, anti-CD94, anti-CD122, anti-CD16, anti-KIR, anti-NKG2A, anti-NKG2D, anti-NKp30, anti-NKp44, anti-NKp46, anti-NKp80. In some embodiments, one or more of the following are used with beads to select regulatory innate lymphoid cells: anti-CD25, anti-CD45, anti-Cd90, anti-CD122, anti-CD127, anti-Sca-1, anti-TGF-βRI, anti-TGF-βRII.

In some embodiments, one or more of the following are used with beads to select macrophage cells: anti-CCR5, anti-CD11a, anti-CD11b, anti-CD11c, anti-CD14, anti-CD15, anti-CD16, anti-CD32, anti-CD33, anti-CD64, anti-CD68, anti-CD80, anti-CD85k, anti-CD86, anti-CD107b, anti-CD115, anti-CD162, anti-EMR1, anti-Galectin-3, anti-GITRL, anti-HLA-DR, anti-MHC-class II, anti-TLR2, anti-TLR4. In some embodiments, one or more of the following are used with beads to select M1 macrophage cells: anti-CD16, anti-CD32, anti-CD36, anti-CD68, anti-CD80, anti-Cd86, anti-HLA-DR, anti-IFNγR, anti-MHC class II. In some embodiments, one or more of the following are used with beads to select M2a macrophage cells: anti-CD163, anti-CD200R1, anti-CD206, anti-CD209, anti-CD301, anti-CXCR1, anti-CXCR2, anti-Dectin-1, anti-HLA-DR, anti-IL-1RII, anti-IL-4Rα, anti-MHC class II. In some embodiments, one or more of the following are used with beads to select M2b macrophage cells: anti-CD86, anti-HLA-DR, anti-IL-4Rα, anti-MHC class II. In some embodiments, one or more of the following are used with beads to select M2c macrophage cells: anti-CCR2, anti-CD150, anti-CD163, anti-IL-4a, anti-CD206, anti-SR-AI, anti-SR-BI, anti-TLR1.

In some embodiments, one or more of the following are used with beads to select monocyte cells: anti-CD11b, anti-CD14, anti-CCR2, anti-HLA-DR, anti-CD115, anti-CD62L. In some embodiments, one or more of the following are used with beads to select intermediate monocyte cells: anti-CD11b, anti-CD14, anti-CD16, anti-CD62L, anti-CD115, anti-CD163, anti-CCR2, anti-CCR5, anti-CX3CR1, anti-HLA-DR. In some embodiments, one or more of the following are used with beads to select non-classical monocyte cells: anti-CD11b, anti-CD14, anti-CD16, anti-CD115, anti-CX3CR1, anti-HLA-DR.

In some embodiments, one or more of the following are used with beads to select monocytic myeloid-derived suppressor cells: anti-CD11b, anti-CD14, anti-CD33, anti-HLA-DR, anti-IL-4Rα. In some embodiments, one or more of the following are used with beads to select polymorphonuclear myeloid-derived suppressor cells: anti-CD11b, anti-CD15, anti-CD16, anti-CD33, anti-CD66b, anti-IL-4Rα, anti-VEGF R1.

In some embodiments, one or more of the following are used with beads to select induced pluripotent stem cells: anti-ABCG2, anti-alkaline phosphatase, anti-Cripto-1, anti-E-Cadherin, anti-Frizzled-5, anti-CD29, anti-CD49f, anti-PODXL, anti-SSEA-3, anti-SSEA-4, anti-TRA-1-60. In some embodiments, one or more of the following are used with beads to select hematopoietic stem cells: anti-ABCG2, anti-CD10, anti-CD34, anti-CD38, anti-CD43, anti-CD44, anti-CD48, anti-CD90, anti-CD93, anti-CD117, anti-CD133, anti-CD150, anti-CDCP1, anti-CXCR4, anti-Flt-3, anti-VEGF R2. In some embodiments, one or more of the following are used with beads to select mesenchymal stem cells: anti-BMP R, anti-CD29, anti-CD44, anti-CD49a, anti-CD51, anti-CD73, anti-CD90, anti-CD105, anti-CD106, anti-CD117, anti-CD166, anti-STRO-1. In some embodiments, one or more of the following are used with beads to select neural stem cells: anti-ABCG2, anti-CD133, anti-CXCR4, anti-FGF R4, anti-Frizzled-9, anti-Glut1, anti-Notch-1, anti-Notch-2, anti-PDGF Ra, anti-SSEA-1.

In exemplary embodiments, the cell modification process is a CAR T cell process. In exemplary embodiments, a CAR T cell modification process includes an isolation step, an activation step, a gene modification step, an expansion step, and a collection step.

In exemplary embodiments, a cell modification system incorporates polymeric microsphere technology to create microspheres specifically engineered to shuttle T cells through the CAR process and maintain the cells in a single containment vessel.

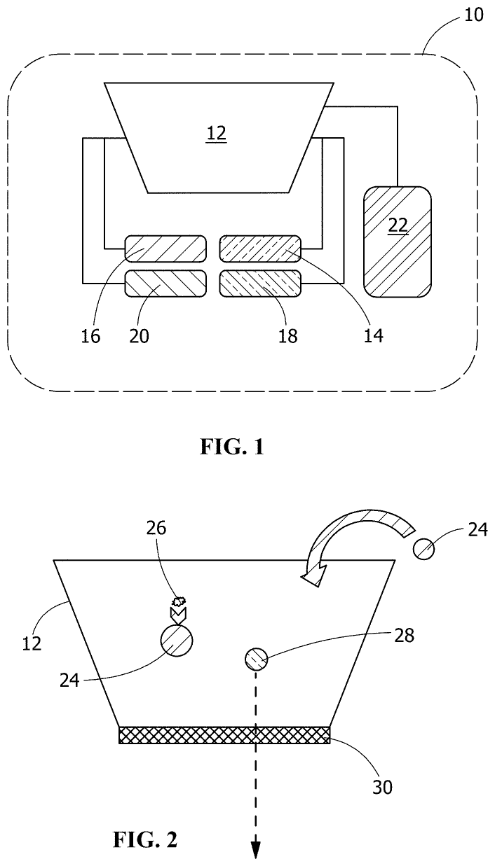

Referring to FIG. 1, the cell modification system 10 includes a cell chamber 12, a first reagent bag 14 including antibody labeled beads for leukapheresis T cell isolation, a second reagent bag 16 including activation beads labeled, for example with anti-CD3 and/or anti-CD28 labeling, a third reagent bag 18 with beads for gene modification, and a fourth reagent bag 20 with expansion beads, and a waste bag 22. Each set of beads are smart beads of PGSU designed for that step of the CAR process. The beads of each type are progressively larger in diameter from the first bag 14 to the second bag 16 to the third bag 18 to the fourth bag 20. The cell modification system 10 is single-use and is loaded into a mainframe that includes a user interface and pumps and other devices to direct fluid flow.

In exemplary embodiments, the T cells shuttle from an antibody-labeled bead to an activation bead to a gene modification bead to an expansion bead during the cell modification process. These beads are of discrete increasing sizes with each set of beads having a tight diameter distribution to permit good separation of one set of beads from the next set of beads. An appropriate diameter distribution for a set of beads is ±50% or less, alternatively ±40% or less, alternatively ±25% or less, alternatively ±10% or less, alternatively ±5% or less, or any value, range, or sub-range therebetween. Appropriate average diameters for the sets of polymeric beads are in the range of about 5 μm to about 1000 μm, alternatively about 10 μm to about 800 μm, alternatively about 10 μm to about 200 μm, alternatively about 20 μm to about 100 μm, or any value, range, or sub-range therebetween. Appropriate diameters for the sets of polymeric beads are in the range of about 5 μm to about 1000 μm, alternatively about 10 μm to about 800 μm, alternatively about 10 μm to about 200 μm, alternatively about 50 μm to about 500 μm, or any value, range, or sub-range therebetween. An appropriate average diameter change between two sets of beads is about 20 μm, alternatively about 20 μm or greater, alternatively about 20 μm to about 50 μm, alternatively about 40 μm to about 60 μm, alternatively about 50 μm or greater, alternatively about 50 μm to about 100 μm, alternatively about 100 μm or greater, or any value, range, or sub-range therebetween.

In exemplary embodiments, a different filter at each separation step separates two sets of beads based on the size of the beads and the size of the pores of the filter. The filters are preferably of a low-protein-binding and low-cell-binding material. In exemplary embodiments, the filters are textile filters, such as, for example, non-degradable, woven textiles. In some embodiments, the textile is an active smart textile, such as, for example, one embedded with a component for the cell modification process, rather than just a passive barrier. In an exemplary embodiment, the active smart textile includes embedded growth factors, such as, for example, interleukins, or includes a sensor embedded in the textile, such as, for example, for glucose monitoring or cell density monitoring. In some embodiments, the textile includes a sheath-core structure with the core being a polyester, for example, and the sheath being dip-coated.

In exemplary embodiments, a cell modification system includes a cell modification chamber including at least one entry port and at least one outlet port. The cell modification system also includes a plurality of filters selectably located on the at least one outlet port, each of the plurality of filters having a predetermined pore size. The cell modification system further includes a plurality of sets of polymeric beads, each set of polymeric beads having a predetermined diameter, the predetermined diameters being selected such that the plurality of sets of polymeric beads are separable by the plurality of filters.

In exemplary embodiments, the cells remain in a single chamber throughout the process. Allowing all processing steps to occur within a single chamber reduces the footprint required to truly make a “bedside” cell modification system. The compact size affords the ability to make CAR a true point-of-care system, allowing for on-site production as opposed to shipment of material between processing facility and treatment center. Microspheres that are tailored for specific cell binding and easily made in various diameters to allow for physical based separations maintain the cells in a central chamber. The cells are not transferred from bag to bag, eliminating bag connections and reducing the possibility of contamination.

The T cells experience reduced shear force as they remain in a central processing chamber. Cell separation is done by size exclusion, eliminating the need for magnetic systems and allowing for higher throughput. The cell chamber may be constantly monitored for all processing parameters and automatically adjusted, as needed. Such monitored parameters may include, but are not limited to, pH, dissolved oxygen levels, and/or dissolved carbon dioxide levels.

In some embodiments, the main chamber is partially filled with media and beads during initial processing, such as, for example, 25% filled, and additional filling occurs during subsequent steps to support activation, transduction, and expansion of the cells. For example, during expansion, the media volume may be increased to 75% and nutrient-loaded beads may then be added so that 50% of the volume is filled by beads. A high media-to-bead ratio allows more space for free-floating T cells to occupy, maximizing process yields.

Additionally or alternatively, the cell chamber may be configured to rely only on gravity for separation of microspheres and products with the filters, eliminating the requirement for pumps and other mechanical systems that can potentially fail.

In exemplary embodiments, T cell selective PGS or PGSU (“Leuka”) beads are used to initially isolate predetermined T cell phenotypes from the leukapheresis product collected from the patient. The Leuka beads are modified with specific antigens to selectively bind the appropriate T cell phenotype and the remaining apheresis material is removed from the chamber through the filter housing below the chamber. The pore size of the step-specific filter is smaller than the diameter of the Leuka beads, keeping them within the cell chamber, thereby isolating the selected T cell phenotype. The chamber and beads may be easily rinsed and cleaned per conventional procedures. This embodiment effectively eliminates the need for a centrifugation step typically required prior to CAR processing.

Referring to FIG. 2, an activation step of the cell modification process begins with adding the appropriate activation media along with activation (“Act”) beads 24 to the cell chamber 12, which already contains the predetermined T cells 26 tethered to Leuka beads 28. The Act beads 24 are modified to promote the appropriate T cell activation.

The T cells 26 tethered to the Leuka beads 28 are cleaved and then allowed to bind to the Act beads 24, and at that point the Leuka beads 28 may be flushed from the chamber through an Act step-specific filter 30 having a pore size greater than the diameter of the Leuka beads 28 but smaller than the diameter of the Act beads 24 such that the Act beads 24 and the tethered T cells 26 remain in the cell chamber 12. The Act step-specific filter 30 is a textile filter.

In some embodiments, the T cells 26 tethered to Leuka beads 28 are cleaved and then allowed to bind to the Act beads 24, and at that point Act beads 24 with tethered T cells 26 are flushed from cell chamber 12 to a secondary container through filter 30 having a pore size larger than Act beads 24 but smaller than Leuka beads 28.

In other embodiments, cell selection and activation take place in the same processing step. In such embodiments, dual isolation and activation occur on the same bead, eliminating the need to have separate Leuka beads and Act beads.

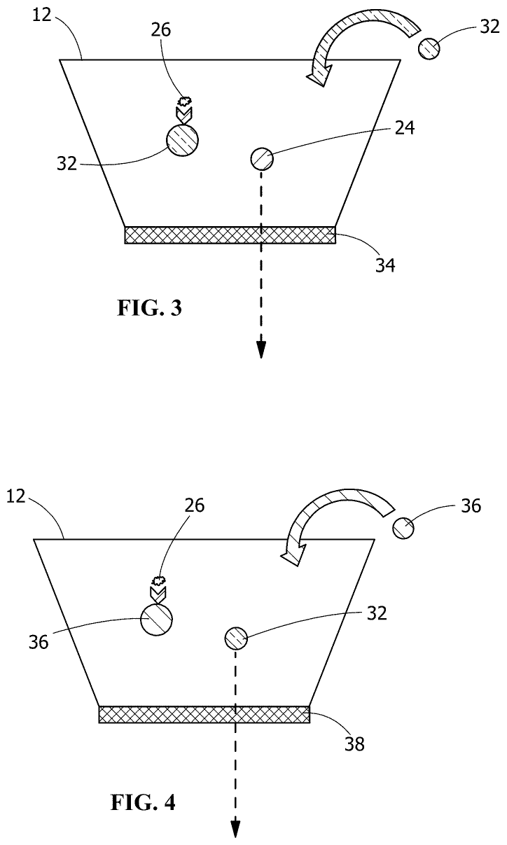

Referring to FIG. 3, a gene modification step of the cell modification process follows a similar procedure to the activation step. The appropriate gene modification media along with gene modification (“GM”) beads 32 is added to the cell chamber 12, which already contains the activated T cells 26 tethered to Act beads 24.

The T cells 26 are cleaved from the Act beads 24 from the Act step and then tethered to the GM beads 32, which allows the Act beads 24 to be removed from the cell chamber through a GM step-specific filter 34 having a pore size greater than the diameter of the Act beads 24 but smaller than the diameter of the GM beads 32 such that the GM beads 32 remain in the cell chamber 12 and the Act beads 24 do not.

In some embodiments, the GM bead 32 is virus-modified. In such embodiments, the virus is tethered to the GM bead 32 with a specific antigen site. This results in increased efficiency in the virus finding a T cell 26, because they are both docking on the same GM bead 32.

In other embodiments, the virus to modify the cells is coupled to much smaller virus beads (not shown) such that the virus coats the virus beads. For example, the Act beads 24 may have a diameter of about 250 μm, and the virus beads may have a diameter of about 25 μm. In exemplary embodiments, the virus bead is a polymeric bead. In some embodiments, the polymeric bead is a PGS bead.

Referring to FIG. 4, an expansion step of the cell modification process also follows a similar procedure to the activation step. The appropriate expansion media along with Expansion (“Exp”) beads 36 is added to the cell chamber 12, which already contains the modified T cells 26 tethered to GM beads 32.

The T cells 26 are cleaved from the GM beads 32 and then tethered to the Exp beads 36, which allows the GM beads 32 to be removed from the cell chamber 12 through an Exp step-specific filter 38 having a pore size greater than the diameter of the GM beads 32 but smaller than the diameter of the Exp beads 36 such that the Exp beads 36 remain in the cell chamber 12 and the GM beads 32 do not.

In some embodiments, the T cells 26 remain on the Act bead 24 for the expansion step. In such embodiments, the cell modification system includes a predetermined number (Y) of Act beads 24, with each Act bead 24 interacting with a maximum number (X) of T cells 26 such that when the total cell count (N) is greater than X×Y, the excess (N−(X×Y)) expanded T cells 26, being unable to be captured on an Act bead 24, are free floating such that the “seed” T cells 26 need not be removed from the Act beads 24. In such embodiments, the cell modification system may include a textile filter on the bottom of the chamber during the expansion step. The textile filter permits free-floating T cells 26 to float down and through but prevents the Act beads 24 from going through.

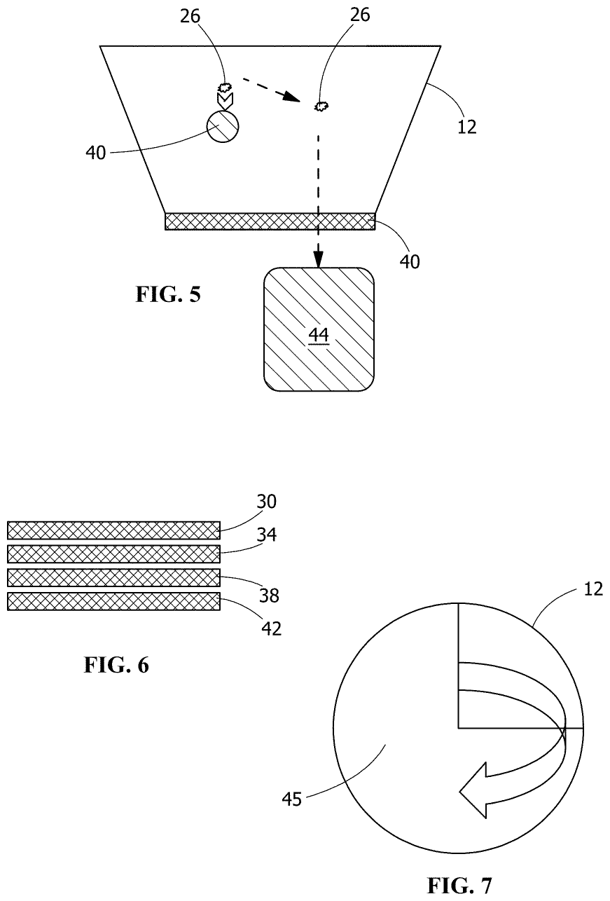

Following the expansion step, the CAR T cells 26 may be cleaned and prepared for cryopreservation in a collection step. Referring to FIG. 5, the T cells 26 are cleaved from the Exp beads 36 and allowed to pass through a collection step-specific filter 42 having a pore size smaller than the diameter of the Exp beads 36 directly into a cryopreservation bag 44, which can be directly removed from the cell modification system 10 and placed into cryopreservation. Alternatively, the collected CAR T cells 26 may be used immediately after collection. In some embodiments, the cryopreservation bag is aseptically connected to the expansion chamber for use of the system in non-sterile environments.

In exemplary embodiments, the cell chamber also houses various processing sensors to monitor certain chamber conditions, such as, for example, pH, O₂ levels, nutrient, CO₂ levels, and/or microbial load, to provide real time data of the chamber condition. Based on the sensor feedback, the chamber environment may be easily adjusted to maintain optimal processing conditions. In exemplary embodiments, O₂ may be bubbled through the mesh filter to aid in cell proliferation. Alternatively, a cell modification system may include a central vertical tube of gas-permeable material running through the chamber to add O₂ and pull out CO₂.

In exemplary embodiments, the polymeric beads are spherical or substantially spherical. In some embodiments, the polymeric beads may be formed to have a non-spherical predetermined shape, such as, for example, a height-to-width ratio of greater than 1, to aid, for example, in filtration selection or processing efficiency.

In some embodiments, a polymeric bead is designed to dissolve during a step of the cell modification process, such as, for example, for delivery of cell nutrients or genetic modification agents. In some embodiments, the dissolution may be triggered by a chamber condition, such as, for example, the pH of the chamber. The condition and the dissolution may be indicative of the process step being complete.

The filters for a cell separation and sorting process or system may be any type appropriate for separation based on particle size. Appropriate filters may include organic membrane filters, inorganic membrane filters, fritted glass filters, and/or textile meshes.

In exemplary embodiments, the filters are textile meshes. Since the filtration mesh is selected for each step, the mesh may be modified with PGS, such as, for example, to provide processing agents for specific steps. In some embodiments, the pore size of a textile mesh layer is generated by laser ablation.

In some embodiments, the filters 30, 34, 38, 42 are stacked vertically, as shown in FIG. 6, where each filter 30, 34, 38, 42 has a different pore size and the remaining filter with the smallest pore size is removed after each filtration.

In some embodiments, the filters 30, 34, 38, 42 are arranged in different quadrants of the cell chamber 12, as shown schematically in FIG. 7, where the filter (not shown) in each quadrant has a different pore size. The filters are oriented parallel to the page in the view of FIG. 7. A mask 45 permits three of the four quadrants to be covered at a given time, and the cell chamber 12 is exposed to a different filter by rotation of the filters or the mask 45. The mask 45 or the filters rotate on an axis perpendicular to the page in the view of FIG. 7.

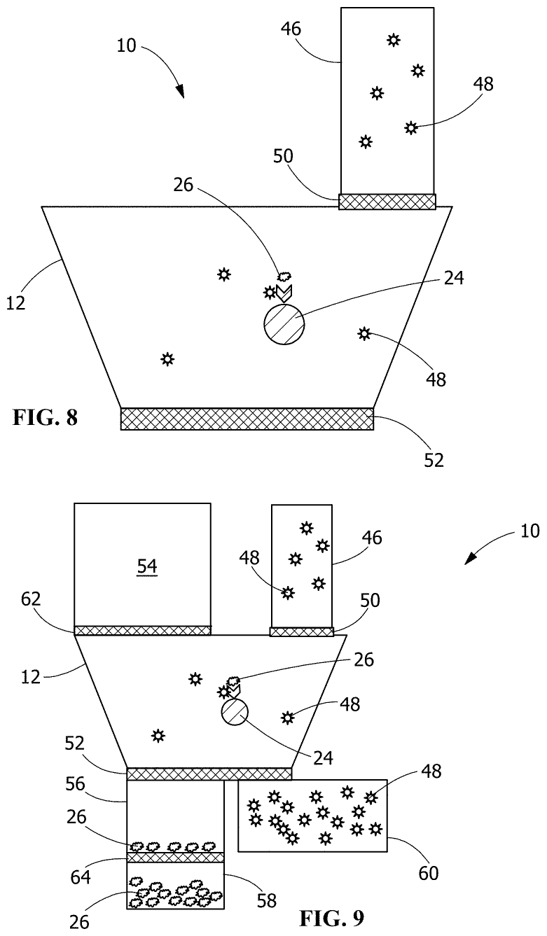

In an exemplary embodiment, a cell modification system 10 includes a small secondary virus antechamber 46, as shown schematically in FIG. 8, where a virus or another gene modification agent is added and attached to or held within the small GM beads 48. The antechamber 46 is separated from the cell chamber 12 by an antechamber port 50, which may be a filter or a valve. A chamber filter 52 is located at another edge of the cell chamber 12.

The contents of the antechamber 46 are washed into the main cell chamber 12 during the activation step. In some embodiments, a sensor (not shown), measuring a change in cytokine level or other soluble identifier, determines when the virus is added to the main cell chamber 12. The antechamber port 50 is kept sterile for plug-and-play functionality outside a biosafety cabinet, and filter or valve separates the genetic modification antechamber 46 and the main chamber.

In exemplary embodiments, a cell modification system 10 is modular and includes an apheresis chamber 54, a virus chamber 46, a main chamber 12, a staging chamber 56, a concentrated CAR T cell collection chamber 58, and a waste chamber 60, as shown schematically in FIG. 9.

The apheresis chamber 54 and the virus chamber 46 each have separate ports 62, 50, respectively, into the main chamber 12. The apheresis product is loaded into the cell modification system 10 by way of the apheresis chamber 54. The virus or other genetic modification agent is loaded into the cell modification system 10 by way of the virus chamber 46. Additional loading units (not shown) may also be located on top of the main chamber 12, such as, for example, to contain beads loaded with nutrient support agents for cell expansion.

The main chamber 12 includes two outlets at the chamber filter 52, one for a waste chamber 60 for collecting waste, such as, for example, washed away virus/GM beads, and one for staging T cells 26 for collection after expansion, and one to the staging chamber 56. The staging chamber 56 has an outlet 64 to the concentrated CAR T cell collection chamber 58. In exemplary embodiments, the cell modification system 10 operates without manual interventions.

The CAR T cells 26 are collected in the concentration chamber 58 for immediate use or freezing after the staging chamber 56 reaches a predetermined CAR T cell 26 population size. The CAR T cells 26 are concentrated in a separate chamber 58, because the T cell concentration in the staging chamber 56 during processing is lower than desired for collection. In exemplary embodiments, the concentration occurs without a centrifuge. In some embodiments, a cryopreservation bag is attached to concentration chamber 58.

In exemplary embodiments, a flow-through cell modification system includes a plurality of catch chambers, each catch chamber having a set of catch polymeric beads of a predetermined diameter to catch a plurality of cells of a predetermined cell type. The flow-through cell modification system also includes a plurality of release chambers, the plurality of catch chambers and the plurality of release chambers being alternatingly sequentially arranged. The flow-through cell modification system further includes a plurality of filters, each filter having a predetermined pore size, each filter separating one of the plurality of catch chambers and one of the plurality of release chambers to separate sets of catch polymeric beads.

In some embodiments, photonic radiation produces the delinking that releases a caught cell from a polymeric bead. To delink by photonic absorption, the target link requires line-of-sight of the emitted photon source because photonic energy propagates in a straight-line. Photonic radiation can be dispersed but cannot propagate around corners beyond the limits of the refractive index interface within through which it passes.

To efficiently delink a mature cell culture, every linked structure should be accessible to the initiating radiation. It may be difficult to ensure that the entire linked population of cell-and-carrier are exposed to the initiating radiation without damaging the culture. A cell culture system may present an optical density problem, where each cell has the potential to eclipse or energetically compete for the delinking photon. Thus, in some embodiments in which a photonic system is used for the catch and release, physical manipulation, such as turbulent mixing of the culture, can be used to expose all linkage absorbing sites.

In other embodiments, remote physical electromagnetic field induced induction, either by a magnetic or electromagnetic energy source such as radio-microwave or magnetic fields, produces the delinking. Under the appropriate field, molecules respond by modifying their electron bonding arrangement or coordination complexation relationships. If one of the associated molecules is paramagnetic or features magnetic susceptibility, the system rearranges in the presence of the induced field like an on-off switch.

In some embodiments, the transition in molecular structure is the classic keto-enol conformational change that may be both photonically and magnetically induced. In some embodiments, the chemistry includes cyanuric acid based on the triazine structure or liquid crystal chemistries and select amino acids with strong dipole functionality and susceptible electron configuration. The field induced delinking reverses the linking association through bonding electron or charge interaction with the inducing field. In some embodiments, amino acids complexed with metals such as manganese have such magnetic susceptibility.

In such embodiments, the entire culture is exposed to the induced field without concern for eclipsing or competition for energy initiation. Such an effect is clearly demonstrated by magnetic resonance imaging (MRI). In exemplary embodiments, an initiating frequency is selected that is low energy and non-ionizing and does not affect the culture.

Additional appropriate release mechanisms to release cells from microspheres may include, but are not limited to, mechanical agitation, cold shock, acidity, competitive binding, microparticle surface erosion, or combinations thereof.

In some embodiments, cells are detached from a PGS particle surface through addition of an agent that competitively binds to the cell ligand site, such as addition of biotin to a media solution containing streptavidin-labeled microspheres that interact with small molecules and proteins, such as antibodies, that have been biotinylated.

In some embodiments, cells are detached from a PGS particle surface through erosion of the PGS surface. In other embodiments, a stimuli sensitive PGS particle is used which dissolves in the presence of media conditions such as presence of enzyme, pH, changes in solution ionic strength.

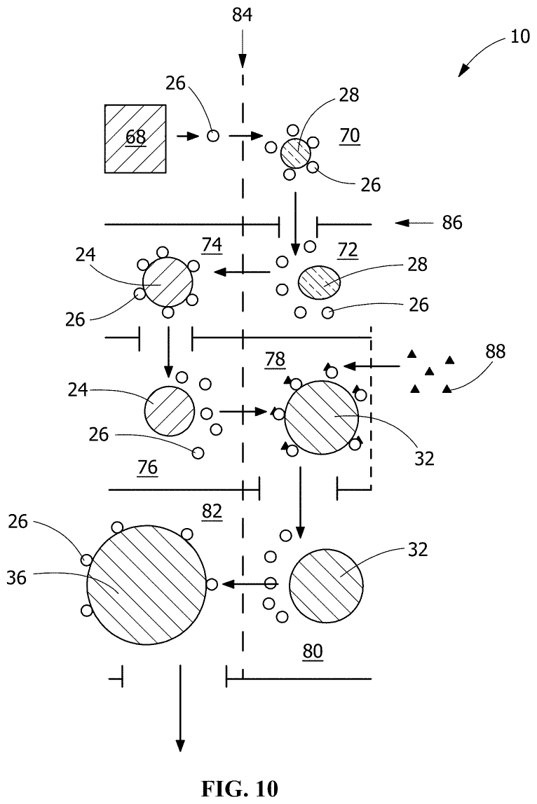

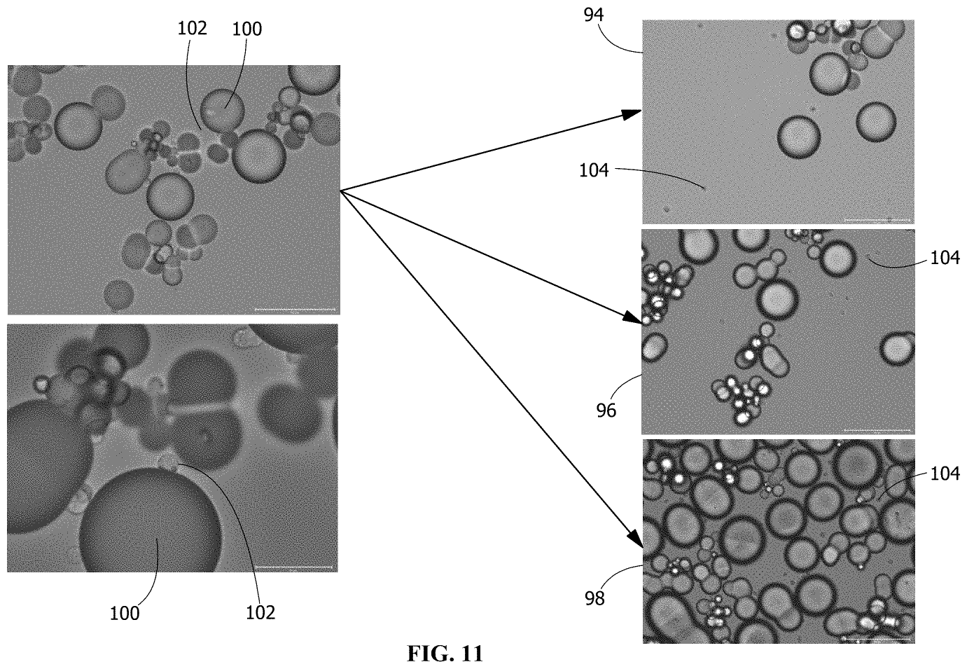

In some embodiments, the cell modification system 10 operates as a flow-through process, as shown schematically in FIG. 10.

Referring to FIG. 10, the cell modification system 10 includes a continuous or semi-continuous mobile-phase “active-nutrient” flow through-process, with the arrows indicating the flow direction, with alternating catch (assembly) chambers 70, 74, 78, 82 and release (sort) chambers 72, 76, 80 requiring size-exclusion microsphere ferrying. The vertical filters 84 in this flow-through process preferably have pores just large enough to permit the target cells to pass through. In some embodiments, the beads are of discrete increasing sizes with each set of beads having a tight diameter distribution to permit good separation of one set of beads from the next set of beads. In some embodiments, the horizontal filters 86 in the above process have pores just large enough for the carrier beads to pass through. As such, the relative size of each set of beads is important to size exclusion, since each set of beads is a ferry to the next chamber. The active nutrient mobile phase may include metabolic support chemistry in combination with the assembly and release biologic “soup”. In other embodiments, the beads are of discrete decreasing size. In other embodiments the beads may be of the same size or with no size trend.

Still referring to FIG. 10, leukocytes 26 pass through a filter to be separated from other formed blood elements (red blood cells and platelets) of blood 68 and into a first catch chamber 70. Leuka beads 28 catch the leukocytes 26 in the first catch chamber 70. The leukocytes 26 travel with the Leuka beads 28 into a first release chamber 72, where they are released from the Leuka beads 28. The leukocytes 26 pass through another filter and into a second catch chamber 74. Act beads 24 catch the leukocytes 26 in the second catch chamber 74. The leukocytes 26 travel with the Act beads 24 into a second release chamber 76, where they are released from the Act beads 28. The leukocytes 26 pass through another filter and into a third catch chamber 78. GM beads 32 catch the leukocytes 26 in the third catch chamber 78 and virus particles 88 are introduced to the leukocytes 26. The leukocytes 26 travel with the GM beads 32 into a third release chamber 80, where they are released from the GM beads 32. The leukocytes 26 pass through another filter and into a fourth catch chamber 82. Exp beads 36 catch the leukocytes 26 in the fourth catch chamber 82. Finally, the leukocytes 72 and Exp beads 36 exit the fourth catch chamber 82.

Subsequent release and catch flow-through segmented sectional units exclusively permit only subject cells having been processed according to the segment and size-restricted PGS microspheres to the next chamber process assembly. Each segmented chamber provides the process step biochemistry and biologic composition to support the associated process step. In some embodiments, each set of bead only shuttles back and forth between its respective catch chamber and release chamber and does not interact with other sets of beads such that the relative size of one set of beads relative to another set of beads is not important.

The pathway of the flow-through system, from start to finish, is a continuous bleed-and-feed chambered assembly that includes the ability to locally isolate each process step for a period of time appropriate to conclude the cell manipulation. Preferably, movement from one chamber volume and content to the next chamber is “temporally” pushed and held for a specified period in which each chamber process is allowed time to develop. For instance, with each fluid volume transfer from one chamber to another, there is a volume holding period until the cells detach from the carriers. During this temporal holding period, the chamber may be temporarily isolated from fluid communication with neighboring chambers. During this time debris and unwanted chemistry may also be eliminated through electrophoresis, electrophoretic action, and chemical scrubbing, including fluid management through local recycling and filtration prior to release of cells to the next chamber. Viable cells from debris may further be directed to selectively pass through the vertical mesh with the aid of an electrophoresis or other active physio electric or physio mechanical device process that distinguishes viable cell related properties from non-living cell and chemical debris. Small molecule contamination may also be similarly separated or filtered or scrubbed using standard commercial chemistries such as Miltenyi Biotec (Bergisch Gladbach, Germany) cellular chemistry. Consequently, each chamber may operate as an in-part bioreactor.

In exemplary embodiments, the size-specific PGS microsphere is the restricted ferry between chambers based on the microsphere size assigned to the respective chamber process assembly event. Movement through to each segmented chamber is restricted by microsphere size-exclusion, whereby each specific size-defined microsphere can only move in one direction and is “restricted” between the catch chamber and the release chamber. Each size-specific microsphere is modified with cell-specific “biologic” tethers to catch cells without applied shear or force.

Only sorted or modified cells pass from one size-specific segmented chamber to another. In some embodiments, the directional passage of cells is limited by the pore size of the chamber wall, depicted as a vertical dashed line 84 in FIG. 10.

Although the steps of the CAR T cell process are described and shown herein as occurring in a specific order, steps of a CAR T cell process may occur in any order. In some embodiments, the process occurs in the order of selection, activation, transduction, expansion, optional selection for product, and collection. In some embodiments, the process may occur with rearrangement of some steps, a partial sequence of steps, or a single step.

Although the cell modification process is specifically described herein in embodiments for CAR T cell formation, the cell modification process may be any process including tethering, release, and separation of cells. In some embodiments, the cell modification process may include bioprocessing immune cells tethered to the bead surface and allowing the tethered cells to produce monoclonal antibodies (mAbs), which are easily removed via a filter and collected, while the tethered cells remain in the chamber.

### EXAMPLES

The invention is further described in the context of the following examples which are presented by way of illustration, not of limitation.

### Example 1

PGSU microspheres for antibody tethering were generated by first melting PGS resin for 1 hour in an oven set to 100° C. Melted resin was then poured into a mixing cup and allowed to cool for 5 minutes before addition of −50% acetone by weight to solubilize the resin. PGS resin and acetone were mixed in a FlackTek mixer (FlackTek, Inc., Landrum, S.C.) for 2 minutes at 2000 RPM to form a homogeneous prepolymer solution. A continuous oil phase was prepared by adding 1200 mL of oil to a 2-L reaction vessel. 36 grams of the surfactant sorbitan trioleate (Span® 85) was added to the reaction vessel and then the impeller, lid, and clamp assembly were constructed to form a seal. Nitrogen gas was then flowed over the continuous oil phase. The impeller was then turned on to mix the continuous oil phase at a speed of about 800 RPM. Optionally, an elevated temperature may be used to increases reaction rate after allowing 30 minutes for the heating mantle to equilibrate thermocouple measurement of reaction vessel temperature. At this point, hexamethylene diisocyanate (HDI) was added to the PGS prepolymer solution and mixed in a FlackTek mixer for 1 minute at 2000 RPM. 14.85 mL of HDI was added to 56 grams of PGS to form a prepolymer with a 3.6:1.0 PGS:HDI ratio. The completed prepolymer solution containing HDI was placed into a 125-mL addition funnel, connected to the reaction vessel, and then metered into the continuous oil phase. PGS microspheres then emulsified and crosslinked for a period of time that was dependent on the temperature of the continuous oil phase but typically in the range of several minutes to 24 hours. Once crosslinking of the microspheres was completed, the PGSU microspheres were extracted from the reaction vessel, washed with heptane to remove residual oil, dried for several days under vacuum, and then sieved to the desired size needed for application.

### Example 2

Sized 45 μm to 75 μm particles of PGSU microspheres were prepared as described in Example 1 for the conjugation of antibodies to the PGSU microsphere surface. For 1-ethyl-3-(3-dimethylaminopropyl)carbodiimide hydrochloride (EDC)/N-hydroxysuccinimide (NETS) based conjugation of antibody to microspheres, 50 mL of activation buffer was prepared by diluting 2-(N-morpholino)ethanesulfonic acid (MES) buffer to 50 mM and adding 0.5 mg/mL sodium dodecyl sulfate (SDS) to act as a surfactant. 500 mg of dried and clean PGSU particles was weighed and placed into a 15 mL centrifuge tube. The surface of the PGSU microspheres was hydroxide etched through addition of a 10 mL solution of 0.1 M NaOH to the microspheres, which were then mixed on a rotary mixer for 5 minutes to ensure even activation of the microsphere surface. After 5 minutes, PGSU microspheres were centrifuged into a pellet at 800 g force for 3 minutes and then the supernatant was removed. Once NaOH was removed, the microspheres were washed three times with 10 mL of activation buffer for 5 minutes, with a centrifugation step at 800 g for 3 minutes and supernatant removal between washes. Following the third wash, microspheres were suspended in 3 mL of the activation buffer and set aside. A solution of EDC/NHS was prepared by weighing out 0.2 g of EDC and 0.2 g of NETS into separate 1.5-mL Eppendorf tubes with 1 mL of activation buffer then added to each tube, which were then vortexed until the EDC and NETS solids were dissolved. The dissolved EDC solution was then added to the tube containing microspheres, followed by addition of the dissolved NETS solution to the tube containing microspheres and EDC. The combined solution was mixed on a rotary mixer for 30 minutes at room temperature and then centrifuged at 800 g for 3 minutes. The supernatant EDC/NHS solution was removed and replaced with 5 mL of activation for a total of two washes for 5 minutes each, with centrifugation and removal of supernatant done at the conclusion of each wash. Activated microspheres were then suspended in 5 mL of phosphate-buffered saline (PBS) buffer at pH 7.4. Conjugation of the antibody was done through addition of 400 μg of antibody to the activated microspheres, which were then mixed on the rotary mixer for 2 hours. Following conjugation, the antibody-bound microspheres were washed three times in 5 mL PBS buffer at pH 7.4 and centrifuged at 800 g for 3 minutes with supernatant removal between wash steps. Following the final wash cycle, microspheres were suspended in 5 mL of PBS buffer to form a 10% w/v solution of PGSU-ab microspheres.

### Example 3

Jurkat cells, an immortalized cell line of a type of human T cells, were seeded at a concentration of 1 million cells/mL for attachment overnight to anti-CD3/anti-CD28 PGSU cell-activation microspheres. The first image 90 and the second image 92 of FIG. 11 show the microspheres 100 and attached cells 102 at high magnification with the scale bar in the first image 90 representing 150 μm and the scale bar in the second image 92 representing 50 μm.

### Example 4

Microspheres 90 with attached Jurkat cells of Example 3 were exposed to mechanical agitation in the form of vigorous pipetting for 2 minutes using a 1 mL pipette. Attached Jurkat cells were released from the cell activation microsphere surface by the mechanical agitation. The third image 94 of FIG. 11 shows cells 104 released by mechanical agitation. The scale bar in the third image 94 represents 150 μm.

### Example 5

Microspheres 90 with attached Jurkat cells of Example 3 were exposed to cold shock in the form of chilled media, specifically media at a temperature of 4° C. for 15 minutes, to harden the cell membrane and release the cells. Attached Jurkat cells were released from the cell activation microsphere surface by the cold shock. The fourth image 96 of FIG. 11 shows cells 104 released by cold shock. The scale bar in the fourth image 96 represents 150 μm.

### Example 6

Microspheres 90 with attached Jurkat cells of Example 3 were exposed to an acidic media, specifically acidic media at a pH of 3.5 for 10 minutes. Attached Jurkat cells were released from the cell activation microsphere surface by the acidic media. The fifth image 98 of FIG. 11 shows cells 104 released by acidic media. The scale bar in the fifth image 98 represents 150 μm.

All above-mentioned references are hereby incorporated by reference herein.

While the invention has been described with reference to one or more exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention but that the invention will include all embodiments falling within the scope of the appended claims. In addition, all numerical values identified in the detailed description shall be interpreted as though the precise and approximate values are both expressly identified.

What is claimed is:

1. A cell selection and sorting process comprising:
attaching cells of a target cell type to a first set of polymeric beads having a first bead diameter in a chamber;
washing the chamber through a first filter having a first pore size less than the first bead diameter to retain the first set of polymeric beads and greater than a cell diameter to remove unattached cells;
releasing the cells of the target cell type from the first set of polymeric beads; and
collecting the cells of the target cell type.

2. The cell selection and sorting process of claim 1, wherein the polymeric beads comprise a co-polymer of glycerol and sebacic acid.

3. The cell selection and sorting process of claim 1, wherein the polymeric beads comprise a surface modification specific to the cells of the target cell type for attachment.

4. The cell selection and sorting process of claim 1, wherein the cells of the target cell type are T cells and the modified cells are chimeric antigen receptor T cells.

5. The cell selection and sorting process of claim 1 further comprising activating the cells of the target cell type prior to washing the chamber.

6. The cell selection and sorting process of claim 1 further comprising transfecting the cells of the target cell type prior to washing the chamber.

7. The cell selection and sorting process of claim 1 further comprising:
after collecting the cells of the target cell type, binding the cells of the target cell type to a second set of polymeric beads having a second bead diameter greater than the first bead diameter;
washing the chamber through a second filter having a second pore size less than the second bead diameter to retain the second set of polymeric beads but greater than the first bead diameter to remove the first set of polymeric beads and unattached cells; and
releasing the cells of the target cell type from the second set of polymeric beads.

8. A cell modification process comprising:
attaching cells of a target cell type to a first set of polymeric beads having a first bead diameter in a chamber;
washing the chamber through a first filter having a first pore size less than the first bead diameter to retain the first set of polymeric beads and greater than a cell diameter to remove unattached cells;
releasing the cells of the target cell type from the first set of polymeric beads;
modifying the cells of the target cell type in the chamber;
binding the cells of the target cell type to a second set of polymeric beads having a second bead diameter greater than the first bead diameter;
washing the chamber through a second filter having a second pore size less than the second bead diameter to retain the second set of polymeric beads but greater than the first bead diameter to remove the first set of polymeric beads;
releasing the modified cells of the target cell type from the second set of polymeric beads; and
collecting the modified cells of the target cell type.

9. The cell modification process of claim 8, wherein the polymeric beads comprise a co-polymer of glycerol and sebacic acid.

10. The cell modification process of claim 8, wherein the polymeric beads comprise a surface modification specific to the cells of the target cell type for attachment.

11. The cell modification process of claim 8, wherein the modifying the cells of the target cell type comprises activating the cells of the target cell type prior to washing the chamber.

12. The cell modification process of claim 8, wherein the modifying the cells of the target cell type comprises transfecting the cells of the target cell type prior to washing the chamber.

13. The cell modification process of claim 8, wherein the cells of the target cell type are T cells and the modified cells are chimeric antigen receptor T cells.

14. A cell modification system comprising:
a cell modification chamber comprising at least one entry port and at least one outlet port;
a plurality of filters selectably located on the at least one outlet port, each of the plurality of filters having a predetermined pore size; and
a plurality of sets of polymeric beads, each set of polymeric beads having a predetermined diameter, the predetermined diameters being selected such that the plurality of sets of polymeric beads are separable by the plurality of filters.

15. The cell modification system of claim 14 further comprising a mask at the at least one outlet port to select one of the plurality of filters for washing the cell modification chamber.

16. The cell modification system of claim 14, wherein the polymeric beads comprise a co-polymer of glycerol and sebacic acid.

17. The cell modification system of claim 14, wherein each of the plurality of sets of polymeric beads comprises a surface modification specific to the cells of the target cell type for attachment.

18. The cell modification system of claim 14, wherein the at least one entry port comprises an apheresis product entry port and a genetic modification agent entry port.

19. The cell modification system of claim 14, wherein the at least one outlet port comprises a cell outlet port and a waste collection port.

20. The cell modification system of claim 19 further comprising a cell staging chamber coupled to the cell modification chamber at the cell outlet port.

21. The cell modification system of claim 14, wherein the cell modification chamber is a single cell modification chamber and all steps of a cell modification process occur in the single cell modification chamber.

22. A flow-through cell selection and sorting system comprising:
a plurality of catch chambers, each catch chamber having a set of catch polymeric beads of a predetermined diameter to catch a plurality of cells of a predetermined cell type;
a plurality of release chambers, the plurality of catch chambers and the plurality of release chambers being alternatingly sequentially arranged; and
a plurality of filters, each filter having a predetermined pore size, each filter separating one of the plurality of catch chambers and one of the plurality of release chambers to separate sets of catch polymeric beads.

23. The flow-through cell selection and sorting system of claim 22, wherein the catch polymeric beads comprise a co-polymer of glycerol and sebacic acid.

24. The flow-through cell selection and sorting system of claim 22, wherein each set of catch polymeric beads comprises a surface modification specific to the cells of the target cell type for attachment.
